(19) United States
(12) Patent Application Publication
(10) Pub. No.: US 2004/0209246 A1
(43) Pub. Date: Oct. 21, 2004

(54) Dual reporter/dye reduction methodology for evaluating antiviral and cytotoxicity of hepatitis C virus inhibitors

(75) Inventors: Mary Brothers, Del Mar, CA (US); Rohit Duggal, San Diego, CA (US); Koleen Herlihy, San Diego, CA (US); Amy Patick, Escondido, CA (US); Weidong Hao, San Diego, CA (US)

Correspondence Address: AGOURON PHARMACEUTICALS, INC., 10350 NORTH TORREY PINES ROAD, LA JOLLA, CA, 92037, US

(73) Assignee: Agouron Pharmaceuticals, Inc.

(21) Appl. No.: 10/818,075

(22) Filed: Apr. 5, 2004

Related U.S. Application Data
(60) Provisional application No. 60/463,245, filed on Apr. 15, 2003.

(51) Int. Cl. C12Q 1/70; C12Q 1/68

(52) U.S. Cl. 435/5

(57) ABSTRACT

Methods are provided for evaluating the antiviral activity and cytotoxicity of a compound in the same population of cells by the use of reporter genes and dye reduction methodology. The dual antiviral activity/cytotoxicity reporter methods are amenable for use in a high-throughput format.

### CROSS-REFERENCES TO RELATED APPLICATIONS

This application claims the benefit of U.S. Provisional application Serial No. 60/463,245, filed Apr. 15, 2003, which is hereby incorporated by reference in its entirety.

### BACKGROUND OF THE INVENTION

Reporter genes are used to identify and analyze regulatory elements of genes. Using recombinant DNA techniques, reporter genes can be fused to a regulatory sequence of interest. The resulting recombinant is then introduced into cells where the expression of the reporter can be detected using various methods, including measurement of: (1) the reporter mRNA; (2) the reporter protein; or (3) the reporter enzymatic activity.

Reporter genes have been used in assays for drug discovery. For example, recombinant cells that express cell surface receptors and that contain reporter-gene constructs responsive to the activity of the cell-surface receptor have been reported for the use of identifying agonists and antagonists of such receptors (see, e.g., U.S. Pat. Nos. 5,401,629; 5,436,128; 5,922,549; and 6,159,705).

Many reporter systems utilize luciferase genes. Luciferase refers to a group of enzymes that catalyze the oxidation of various substrates to produce a light emission. Generally, since luciferase activity is not found in eukaryotic cells, it is advantageous to use luciferase for studying promoter activity in mammalian cells. The most popular luciferases for use as reporter genes are the bacterial luciferases, the firefly (Photinus pyralis) luciferase, the Aequorin luciferase and more recently the Renilla luciferase.

The wild-type luciferase enzyme of the sea pansy Renilla reniformis catalyzes the emission of visible light in the presence of oxygen and coelenterazine to produce blue light. The luciferase gene from Renilla has been used to assay gene expression in bacterial (Jubin et al., Biotechniques 24:185-188 (1998)), yeast (Srikantha et al., J. Bacteriol. 178:121-129 (1996)), plant (Mayerhofer et al., Plant J. 7:1031-1038 (1995)), and mammalian cells (Lorenz et al., J. Biolumin. Chemilumin. 11:31-37 (1996)).

Multiple assay formats are a known tool for evaluating the potential antiviral activity of putative inhibitors. Common antiviral assay methods include quantitatively measuring the production of viral antigens and the activities of viral enzymes as indicators of virus replication. Although highly sensitive, these methods are often cumbersome and difficult to format for high-throughput screening. Alternatively, virus replication can be measured indirectly by monitoring viral-induced host-cell cytopathic effects using dye reduction methods (cell protection assays), which are simple and can usually be adapted for medium- to high-throughput analyses. However, cell-protection assays are limited to highly lytic virus replication systems and often require lengthy assay timeframes (typically at least 4 days).

One component of a cell-based drug-screening assay is assessing a test compound's cytotoxicity or specificity. Cytotoxicity measurements, when combined with compound activity data, elucidate specific compound activity from non-specific inhibitor effects or cytotoxicity. In fact, for the majority of the assays described in the art, including reporter virus assays, a separate assay format must be used to evaluate inhibitor-mediated cytotoxicity. In cell protection assays, antiviral and cytotoxic effects of an inhibitor can be measured using the same method; however, accurate evaluations of antiviral and cytotoxic activities must be performed in separate assays (i.e., separate cell populations). Therefore, antiviral screens using the existing assay formats must include a separate counterscreen to evaluate a compound's cytotoxicity, which requires significant resources and considerable reductions in overall screen throughput.

A commercially available reporter system (Dual-Luciferase® Reporter Assay System) is available from Promega Corporation. It first measures firefly luciferase activity followed by measurement of Renilla luciferase activity (see U.S. Pat. No. 6,171,809). Dual measurement of firefly and Renilla luciferase activity in transfected cells is described in U.S. Pat. Nos. 6,261,791, 6,235,873, 6,255,112, 6,255,473, 6,143,502 and 6,063,578. Single and dual reporter assays based on luciferase activity are also discussed in International Publication WO96/40988. However, none of these single and dual reporter assays comprises a sensitive antiviral assessment along with an integrated cytotoxicity assessment.

Hepatitis C virus (HCV) is a member of the hepacivirus genus in the family Flaviviridae. It is the major causative agent of non-A, non-B viral hepatitis and is the major cause of transfusion-associated hepatitis and accounts for a significant proportion of hepatitis cases worldwide. HCV is an enveloped ribonucleic acid (RNA) virus containing a single-stranded positive-sense RNA genome approximately 9.5 kb in length [Choo et al., Science 244:359-362 (1989)].

The development of small-molecule inhibitors directed against specific viral targets has become a focus of anti-HCV research. However, the expedited identification and development of such inhibitors relies on the use of efficient, high-throughput drug-screening assays. Therefore, there is continual need to develop more rapid and efficient drug-screening assays, particularly in the antiviral field.

### SUMMARY OF THE INVENTION

The present invention is directed generally to a dual reporter assay combined with a dye reduction method that facilitates evaluation of anti-hepatitis C virus (HCV) activity and cytotoxicity of compounds in the same population of cells. The dual reporter assay is amenable to a high-throughput format, and may be utilized in HCV drug discovery activities.

Described herein are methods for evaluating antiviral activity and cytotoxicity of a compound comprising providing a target cell population containing a first reporter gene; introducing a second reporter gene into the cell population by integrating the reporter into a replicon of a positive strand RNA virus and making a dual reporter replicon cell line; adding a test compound; incubating the cell population; measuring the responses of the first and second reporter genes; and comparing the responses of the first and second reporter genes in cell populations treated with compound to the responses of the first and second reporter genes in cell populations in the absence of the compound.

In a preferred embodiment, a method of evaluating antiviral activity and cytotoxicity of a compound is provided wherein the response from the first reporter gene indicates a measure of cell viability, the response from the second reporter gene indicates the activity of a virus, and the second reporter gene is different from the first reporter gene.

In another preferred embodiment, a method of evaluating antiviral activity and cytotoxicity of a compound is provided wherein the first reporter gene comprises firefly luciferase and the second reporter gene comprises humanized Renilla reneformis luciferase.

In another preferred embodiment, a method of evaluating antiviral activity and cytotoxicity of a compound is provided wherein the first reporter gene comprises humanized Renilla reneformis luciferase and the second reporter gene comprises firefly luciferase.

In another preferred embodiment, a method of evaluating antiviral activity and cytotoxicity of a compound is provided wherein the compound being evaluated comprises an HCV inhibitor.

In another preferred embodiment, a method of evaluating antiviral activity and cytotoxicity of a compound is provided wherein the response of the second reporter gene indicates the activity of an RNA virus.

In another preferred embodiment, the cell population is selected from Huh-7 cells; HeLa cells; VERO cells; CHO cells; COS cells; BHK cells; HEPG2 cells; 3T3 cells and 293 cells.

In still another preferred embodiment, the method is performed wherein the cell population is contained within a configuration of low-throughput, medium-throughput or high-throughput screening wells.

In yet another preferred embodiment, a method is provided for evaluating antiviral activity and cytotoxicity of a compound comprising providing a target cell population containing a first reporter gene; introducing a second reporter gene into the cell population by integrating the reporter into a replicon of a positive strand RNA virus and making a dual reporter replicon cell line; adding a test compound; incubating the cell population; adding a dye reduction agent to the cell population; measuring the response from the reduced dye reduction agent in the cell population; comparing the response of the reduced dye reduction agent in the compound treated cell population to the response of the reduced dye reduction agent in the cell population in the absence of the test compound; measuring the expression of the first and second reporter genes; and comparing the responses of the first and second reporter genes in the cell populations treated with compound to the responses of the first and second reporter genes in the cell populations in the absence of the compound.

In another preferred embodiment, the invention comprises a double-transformed mammalian cell line wherein a first transformation is due to integration of a gene in the nucleus and a second transformation is due to the replication of a RNA virus replicon.

In another preferred embodiment, the invention comprises a cell line wherein the first and second transformations incorporate the expression of reporter genes to produce a double-stable, double-reporter cell line.

In another preferred embodiment, the invention comprises a cell line wherein the second transformation is due to the replication of an HCV replicon.

In another preferred embodiment, the invention comprises a transformed cell line comprising a reporter construct wherein the construct expresses a marker for monitoring cytotoxicity.

In yet another preferred embodiment, a method is provided for evaluating both antiviral activity and cytotoxicity of a compound in the same population of cells comprising the steps of providing a target cell comprising a reporter gene that is indicative of the activity of an HCV or other RNA virus replicon; adding a test compound to the population of cells; incubating the cell population; adding a dye reduction agent to the cell population; measuring the response from the reduced dye reduction agent in the cell population; comparing the response of the reduced dye reduction agent in a compound treated cell population to the response of the reduced dye reduction agent in cell populations in the absence of the compound; measuring the expression of the reporter gene integrated in the replicon; and comparing the responses of the reporter gene of the replicon in cell populations treated with compound to the responses of the reporter gene of the replicon in cell populations in the absence of the compound.

In yet another preferred embodiment, a method is provided for generating a double-stable, double-reporter cell population comprising integrating a reporter gene into the cell nuclei of the cell population and introducing a replicating RNA virus sequence, including an HCV sequence, into the cell population.

In another preferred embodiment, a kit is provided for carrying out an assay for evaluating both antiviral activity and cytotoxicity of a compound in the same population of cells comprising in packaged combination: (a) a target cell comprising a reporter gene that is indicative of the activity of an HCV or other RNA virus replicon; (b) standard control and dye reduction reagents; and (c) instructions for carrying out the assay. The kit can also contain, depending on the particular methodology employed, suitable labels and other packaged reagents and materials (i.e. wash buffers and the like). Standard screening assays, such as those described above, can be conducted using these kits.

Preferred reporter genes for use in the assays described herein are firefly luciferase and humanized Renilla reneformis luciferase (Promega Corp., Madison, Wis.) genes. In another embodiment of the invention, dye reduction methodology can be applied to the dual reporter system in suitable host cells. Examples of suitable host cells are Huh-7 cells, HeLa cells, VERO cells, CHO cells, COS cells, BHK cells, HEPG2 cells, 3T3 cells, or 293 cells.

### BRIEF DESCRIPTION OF THE DRAWINGS

FIG. 1 depicts a schematic of the HCV genome and the HCV hRLuc-selectable replicon construct (BB7M4hRLuc). The 5′ and 3′ nontranslated regions (NTRs) flank the open reading frame with the structural proteins located in the NH₂-terminal portion of the polyprotein. The remainder encodes the nonstructural proteins (NS2 to NS5B). The reporter-selectable replicon, designated BB7-M4-hRLuc, has the 5′ NTR fused to a small portion of the core coding region, the humanized Renilla luciferase gene (hRLuc), a self-cleaving peptide of foot and mouth disease virus (FMDV) 2A proteinase, the NPTII gene, and an EMCV IRES (designated “El”), followed by the NS3 to NS5B HCV coding region and the 3′ NTR region.

FIG. 2 provides the sequence for the pcDNA6.Fluc reporter construct.

FIG. 3 is a schematic diagram for Huh-7 cells of the dual reporter replicon cell line, B6b, which shows the hRLuc reporter selectable replicon producing hRLuc to monitor antiviral activity, and the integrated FLuc gene in the nucleus to monitor cytotoxicity.

FIG. 4 shows the use of the Z′ factor to demonstrate the quality of the dual reporter assay. The formula for Z′ factor takes into consideration the signal to noise ratio as well as the variation in the assay. Z′ values between 1-0.5 are indicative of a robust assay that can be used for carrying out high throughput screening.

### DETAILED DESCRIPTION OF THE INVENTION

The invention pertains to a dual reporter assay combined with a dye reduction method that facilitates evaluation of anti-hepatitis C virus (HCV) activity and cytotoxicity of compounds in the same population of cells. The assay is amenable to low-, medium- and high-throughput formats, and may be utilized in HCV drug discovery activities.

In accordance with the present invention there may be employed conventional molecular biology, microbiology, and recombinant DNA techniques within the skill of the art. Such techniques are explained fully in the literature. See, e.g., Maniatis et al., “Molecular Cloning: A Laboratory Manual,” (1989); Ausubel, Ed., “Current Protocols in Molecular Biology,” Volumes I-III (1994); Celis, Ed., “Cell Biology: A Laboratory Handbook,” Volumes I-III (1994); Coligan, Ed., “Current Protocols in Immunology,” Volumes I-III (1994); Gait, Ed., “Oligonucleotide Synthesis” (1984); Hames et al., Eds., “Nucleic Acid Hybridization” (1985); Hames et al., “Transcription and Translation” (1984); Freshney, Ed., “Animal Cell Culture” (1986); IRL Press, “Immobilized Cells and Enzymes” (1986); and Perbal, “A Practical Guide To Molecular Cloning” (1984).

As used herein, the terms “comprising” and “including” are used in an open, non-limiting sense. Further, if appearing herein, the following terms shall have the definitions set forth below.

“Polynucleotide” or “nucleic acid molecule” generally refers to any polyribonucleotide or polydeoxribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

In addition, the term “DNA molecule” refers only to the primary and secondary structure of the molecule, and does not limit it to any particular tertiary forms. Thus, the term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “RNA molecule” refers to the polymeric form of ribonucleotides in its either single-stranded form or a double-stranded helix form. In discussing the structure of particular RNA molecules, sequence may be described herein according to the normal convention of giving the sequence in the 5′ to 3′ direction.

Amino acid residues described herein are preferred to be in the “L” isomeric form. However, residues in the “D” isomeric form can be substituted for any L-amino acid residue, as long as the desired functional property is retained by the polypeptide.

The term NH₂ refers to the free amino group present at the amino terminus of a polypeptide, while COOH refers to the free carboxy group present at the carboxy terminus of a polypeptide. Standard polypeptide nomenclature and abbreviations for amino acid residues are used herein.

Amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues.

A “replicon” is any genetic element (e.g., plasmid, chromosome, viral RNA) that functions as an autonomous unit of DNA or RNA replication in vivo. That is, it is capable of replication under its own control. Bradenbeck et al., Semin. Virol. 3:297-310 (1992).

A “vector” is a circular DNA, such as a plasmid, phage or cosmid, to which another DNA segment may be attached so as to bring about the replication, expression or integration of the attached segment.

A variety of expression vectors can be used to express a nucleic acid molecule. Such vectors include chromosomal, episomal, and virus-derived vectors, e.g., vectors derived from bacterial plasmids, from bacteriophage, from yeast episomes, from yeast chromosomal elements, including yeast artificial chromosomes, from viruses such as baculoviruses, papovaviruses such as SV40, vaccinia viruses, adenoviruses, poxviruses, pseudorabies viruses, herpes viruses, and retroviruses. Vectors may also be derived from combinations of these sources, such as those derived from plasmid and bacteriophage genetic elements, e.g., cosmids and phagemids. Appropriate cloning and expression vectors for prokaryotic and eukaryotic hosts are described in Sambrook et al., supra.

A vector containing the appropriate nucleic acid molecule can be introduced into an appropriate host cell for propagation or expression using known techniques. Host cells can include bacterial cells including, but not limited to, E. coli, Streptomyces, and Salmonella typhimurium, eukaryotic cells including, but not limited to, yeast, insect cells, such as Drosophila, animal cells, such as Huh-7, HeLa, COS, HEK 293, MT-2T, CEM-SS, and CHO cells, and plant cells.

Vectors generally include selectable markers that enable the selection of a subpopulation of cells that contain the recombinant vector constructs. The marker can be contained in the same vector that contains the nucleic acid molecules described herein or may be on a separate vector. Markers include tetracycline- or ampicillin-resistance genes for prokaryotic host cells and dihydrofolate reductase or neomycin resistance for eukaryotic host cells. However, any marker that provides selection for a phenotypic trait will be effective.

A “coding sequence” or “open reading frame” is a nucleotide sequence that is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA or RNA sequences.

Transcriptional control sequences are DNA regulatory sequences, such as promoters, enhancers, polyadenylation signals, terminators, and the like, that provide for the expression of a coding sequence in a host cell by synthesis of messenger RNA (mRNA) from the DNA template.

A “promoter sequence” is a DNA regulatory region capable of binding RNA polymerase in a cell and initiating transcription of a coding sequence. For purposes of defining the present invention, a promoter sequence is bound at its 3′ terminus by the transcription initiation site and extends upstream (5′ direction) to include the minimum number of bases or elements necessary to initiate transcription at levels detectable above background. Within the promoter sequence will be found a transcription initiation site, conveniently defined by mapping with nuclease S1, as well as protein binding domains (consensus sequences) responsible for the binding of RNA polymerase. Prokaryotic promoters contain −10 and −35 consensus sequences. A promoter can also be used to refer to RNA sequences or structures in RNA virus replication.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence. RNA sequences can also serve as expression control sequences by virtue of their ability to modulate translation, RNA stability, and replication (for RNA viruses).

A “signal sequence” can be included before the coding sequence. This sequence encodes a signal peptide, N-terminal to the polypeptide, which communicates to the host cell to direct the polypeptide to the cell surface or secrete the polypeptide into the media. The signal peptide is clipped off by the host cell before the protein leaves the cell. Signal sequences can be found associated within a variety of proteins native to eukaryotes.

The term “oligonucleotide” is defined as a molecule comprised of two or more deoxyribonucleotides, preferably more than three. Its exact size will depend upon many factors, which, in turn, depend upon the ultimate function and use of the oligonucleotide.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced. That is, inducement in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. The primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby carry out the synthesis of the extended product.

As used herein, the terms “restriction endonucleases” and “restriction enzymes” refer to bacterial enzymes, each of which cut double-stranded DNA at or near a specific nucleotide sequence.

A cell has been “transformed” by exogenous or heterologous DNA or RNA when such DNA or RNA has been introduced inside the cell. The transforming DNA or RNA may or may not be integrated (covalently linked) into chromosomal DNA making up the genome of the cell. For example, in prokaryotes, yeast, and mammalian cells, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. In the case of an RNA replicon that transforms a mammalian cell as described in the present invention, the RNA molecule, e.g., an HCV RNA molecule, has the ability to replicate semi-autonomously. Huh-7 cells carrying the HCV replicons get selected in the presence of G418 since HCV RNA replication results in resistance to G418 by production of the neomycin phosphotransferase protein. This results in clones of Huh-7 cells resistant to G418, which are capable of forming cell lines. These clones of cells can be further transformed/transduced with expression vectors, such as the one that carries the firefly luciferase gene (pcDNA6.Fluc) to generate stable cell lines that require selection by two antibiotic markers.

A “clone” is a population of cells derived from a single cell or common ancestor by mitosis.

The term “recombinant host cell” refers to a cell that has been altered to contain a new combination of genes or nucleic acid molecules. The recombinant host cells were prepared by introducing the vector constructs described herein into the cells by techniques readily available in the art. These include calcium phosphate transfection, DEAE-dextran-mediated transfection, cationic lipid-mediated transfection, electroporation, transduction, infection, lipofection, and other techniques, such as those found in Sambrook et al., “Molecular Cloning: A Laboratory Manual” (2001).

Host cells can contain more than one vector. Thus, different nucleotide sequences can be introduced on different vectors to the same cell. Similarly, the nucleic acid molecules can be introduced either alone or with other nucleic acid molecules that are not related to the nucleic acid molecules, such as those providing trans-acting factors for expression vectors. When more than one vector is introduced into a cell, the vectors can be introduced independently, co-introduced or segments of each vector can be combined into one vector. The invention also relates to recombinant host cells containing the vectors described herein.

In the case of bacteriophage and viral vectors, these can be introduced into cells as packaged or encapsulated virus by standard procedures for infection and transduction. Viral vectors can be replication-competent or replication-defective. In the case in which viral replication is defective, replication will occur in host cells providing functions that complement the defects.

A “cell line” is a clone of a primary cell that is capable of stable growth in vitro for many generations. RNA or DNA molecules, which can be used to transform or “transfect” cells can be used for making transformed cell lines. For some RNA viruses, such methods can be used to produce cell lines which transiently or continuously support virus replication and, in some cases, which produce infectious viral particles.

Two DNA or RNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al, supra.

A “heterologous” region of a DNA or RNA construct is an identifiable segment of DNA or RNA molecule within a larger nucleic acid that is not found in association with the larger molecule in nature. For instance, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally occurring mutational events do not give rise to a heterologous region of DNA as defined herein.

A DNA sequence is “operatively linked” to an expression control sequence when the expression control sequence controls and regulates the transcription and translation of that DNA sequence. The term “operatively linked” includes having an appropriate start signal (e.g., ATG or AUG) in front of the DNA sequence to be expressed and maintaining the correct reading frame to permit expression of the DNA sequence under the control of the expression control sequence and production of the desired product encoded by the DNA sequence. If a gene to be inserted into a recombinant DNA molecule does not contain an appropriate start signal, such a start signal can be inserted in front of the gene.

The term “standard hybridization conditions” in general refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of standard hybridization conditions is whether the two sequences hybridizing are RNA-RNA, DNA-DNA or RNA-DNA. Such standard hybridization conditions are easily determined by one skilled in the art according to well-known formulae, wherein hybridization is typically 10-20° C. below the predicted or determined Tₘ.

As used herein, “ng” means nanogram, “ug” or “μg” mean microgram, “mg” means milligram, “ul” or “μl” mean microliter, “μF” means micro Farraday, “ml” means milliliter, “I” means liter, “min.” means minutes and “sec.” means seconds.

Hepatitis C virus or HCV refers to a diverse group of related viruses classified as a separate genus in the Flaviviridae family. The characteristics of this genus are described in the Background of the Invention above, and include such members as HCV-1, HC-J1, HCV-J, HCV-BK, HCV-H, HC-J6, HC-J8, HC-J4/83, HC-J4/91, HC—C2, HCV-JK1, HCV-T, HCV-JT, HC-G9, and the like.

HCV analogs may be prepared from nucleotide sequences derived within the scope of the present invention. Analogs, such as fragments or mutants can be produced by standard cleavage by restriction enzymes, or site-directed mutagenesis of the HCV coding and non-coding (5′ and 3′ terminal) sequences. Molecules exhibiting “HCV inhibiting activity” such as small molecules, cytokines or antisense molecules may be identified by assays, e.g., using interferon.

Replication of HCV in cells can be ascertained by branched TaqMan quantitative RT/PCR and immunological procedures. The procedures and their application are well known in the art and accordingly may be utilized within the scope of the present invention. A “competitive” antibody binding procedure is described in U.S. Pat. Nos. 3,654,090 and 3,850,752. A “sandwich” procedure is described in U.S. Pat. Nos. RE 31,006 and 4,016,043. Still other procedures are known such as the “double antibody”, or “DASP” procedure.

In each instance, HCV proteins form complexes with one or more antibodies or binding partners and one member of the complex is labeled with a detectable label. The fact that a complex has formed and, if desired, the amount thereof, can be determined by known methods applicable to the detection of labels.

Alternatively, the presence of HCV RNA can be determined by Northern analysis, primer extension, and the like. The labels most commonly employed for these studies are radioactive elements, enzymes that fluoresce when exposed to substrate and others. A number of fluorescent materials are known and can be utilized as labels. These include, for example, fluorescein, rhodamine, auramine, Texas Red, AMCA blue and Lucifer Yellow.

An antibody to HCV proteins or a probe for HCV RNA can also be labeled with a radioactive element or with an enzyme. The radioactive label can be detected by any of the currently available counting procedures. The preferred isotope may be selected from ³H, ¹⁴C, ³²P, ³⁵S, Cl, ⁵¹Cr, ⁵⁷Co, ⁵⁸ Co, ⁵⁹Fe, ⁹⁰Y, ¹²⁵I, ¹³¹I, and ¹⁸⁶Re.

Enzyme labels are likewise useful, and can be detected by any of the presently utilized colorimetric, spectrophotometric, fluorospectrophotometric, techniques. The enzyme is conjugated to the selected probe by reaction with bridging molecules such as carbodiimides, diisocyanates, glutaraldehyde and the like. Many enzymes that can be used in these procedures are known and can be utilized. Those preferred are peroxidase, beta-glucuronidase, beta-D-glucosidase, beta-D-galactosidase, urease, glucose oxidase plus peroxidase and alkaline phosphatase. U.S. Pat. Nos. 3,654,090; 3,850,752; and 4,016,043 are referred to by way of example for their disclosure of alternate labeling material and methods. In addition, a probe may be biotin-labeled, and thereafter be detected with labeled avidin, or a combination of avidin and a labeled anti-avidin antibody. Probes may also have digoxygenin incorporated therein and be then detected with a labeled anti-digoxygenin antibody.

An EC₅₀ value is the concentration of the inhibitor at which 50% inhibition of viral replication is achieved. An HCV replicon reporter assay system can be developed to determine the specific antiviral activity of inhibitors in standard dose response assays. In such assays, the reporter-selectable containing Huh-7 cells are incubated in 96 wells containing serial dilutions of test inhibitors or no inhibitor. At a specified time after incubation, the activities of the viral-encoded reporter genes are measured in the cell lines using the appropriate reporter assay methodologies. Data from the reporter gene measurements can be expressed as the percent of reporter gene activity in inhibitor-treated cells relative to that of inhibitor-free cells. An analysis of the antiviral component of such data allows for the calculation of the fifty-percent effective concentration (EC₅₀). Similarly, an EC₉₀ value is the concentration of the inhibitor at which 50% inhibition of viral replication is achieved, and an analysis of the antiviral component of a data set allows for a calculation of the ninety-percent effective concentration (EC₉₀).

A CC₅₀ value is the concentration of the inhibitor at which 50% cell death has occurred. In the current invention, two end points will be used to measure CC₅₀ values. The first is XTT dye reduction methodology and the second is the use of the second reporter gene integrated in the nucleus of cells carrying the reporter-selectable HCV replicons. In case of both methodologies, a CC₅₀ value will be generated from the same wells from which an EC₅₀ value was obtained. The CC₅₀ value would be generated by calculating % cytotoxicity of the inhibitor/compound treated wells compared to the no inhibitor/compound well resulting in generation of a dose dependent curve to obtain the value that was responsible for the death of 50% of the cells.

An internal ribosomal entry site (IRES) recruits ribosomes in a cap-independent manner to carry out translation. The HCV RNA genome contains an internal ribosome entry site.

A “BB7” construct (obtained from Apath, L.L.C., St. Louis, Mo.) is a subgenomic HCV RNA (replicon) having one adaptive mutation (S2204I) in the NS5A domain.

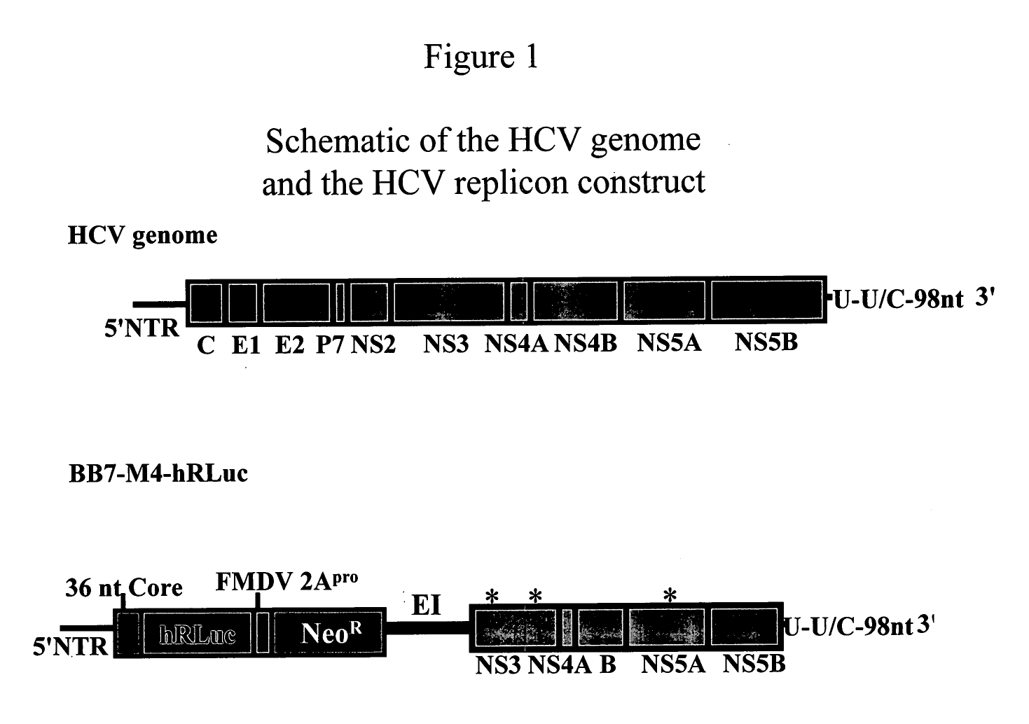

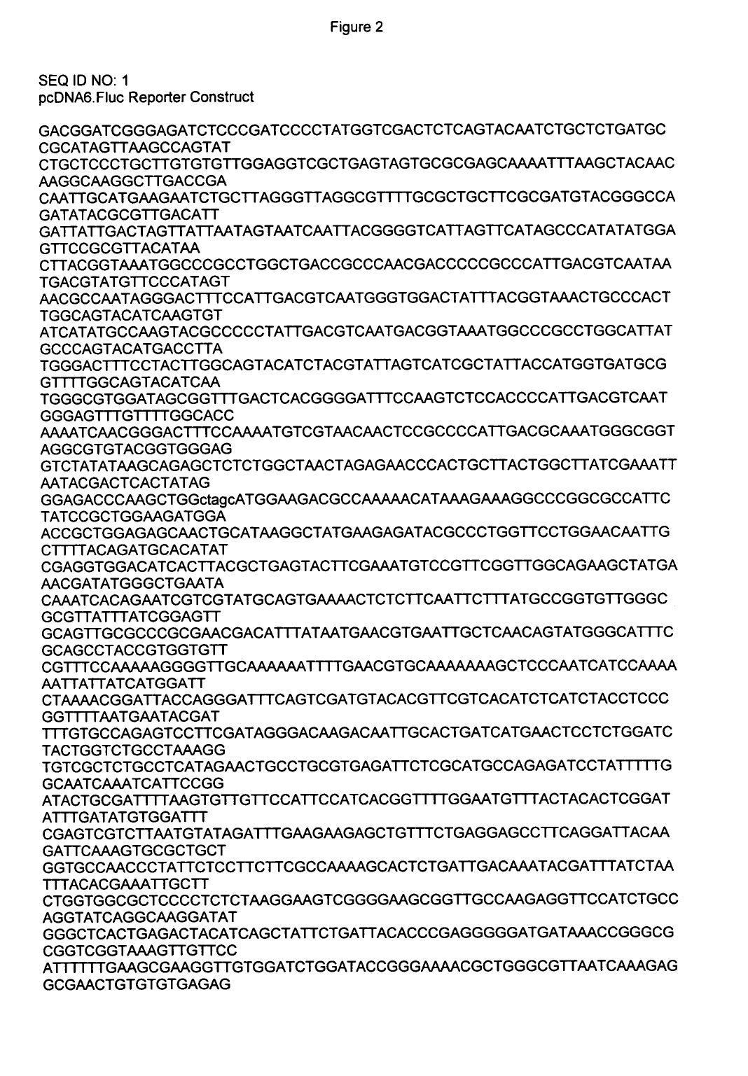

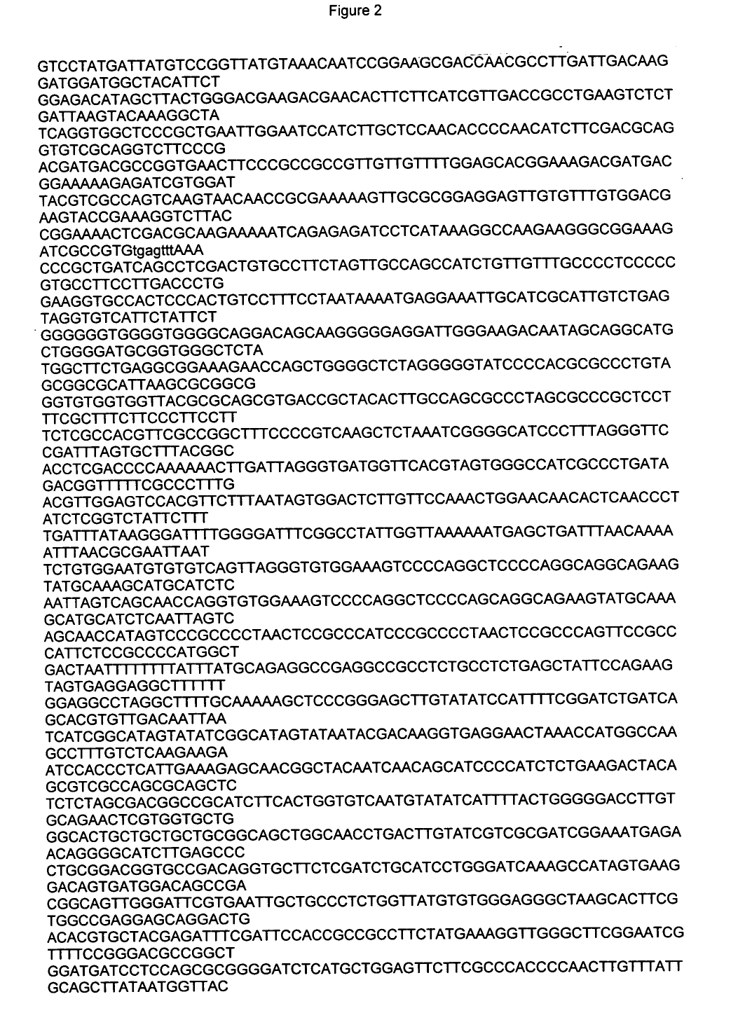

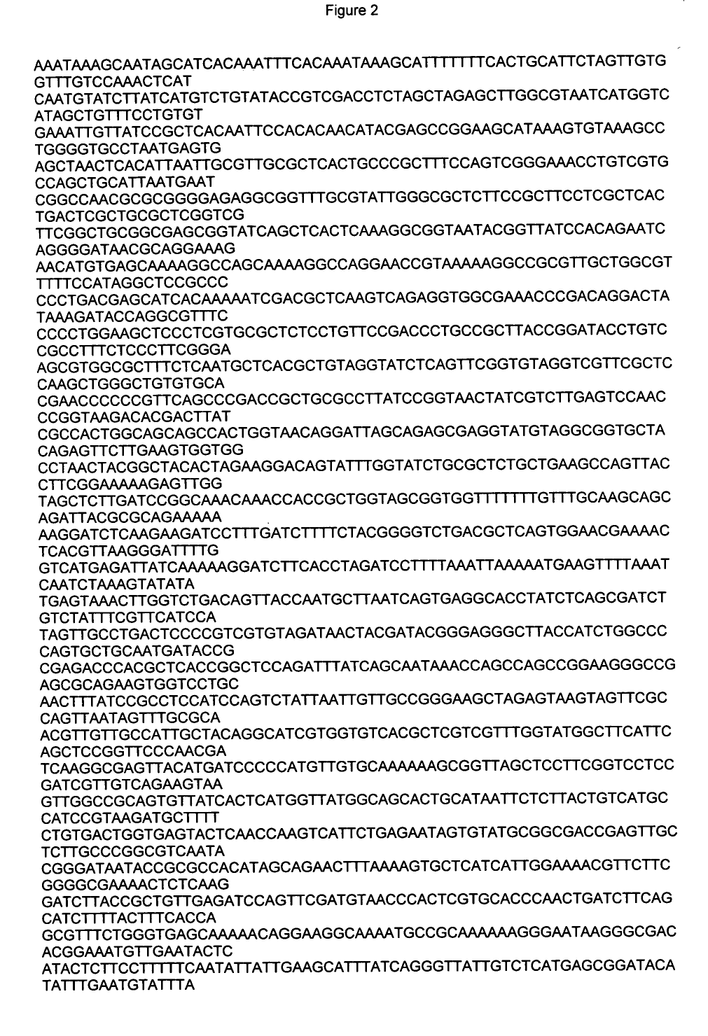

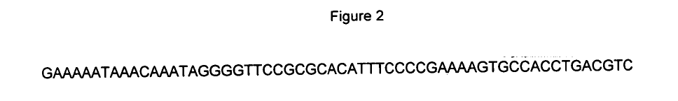

The reporter-selectable HCV replicon, which may be designated herein BB7M4hRLuc, has a reporter gene to monitor HCV replication and three adaptive mutations, two in the NS3 domain and one in the NS5A domain (FIG. 1). The reporter gene (hRLuc) is fused to the NPT II gene via a self-cleaving peptide encoding the 2A proteinase of FMDV. This fusion protein is under the translational control of the HCV IRES residing in the 5′ nontranslated region (NTR) of HCV RNA. The second cistron of the replicon that comprises the HCV nonstructural protein region from NS3-NS5B is under the translational control of EMCV IRES.

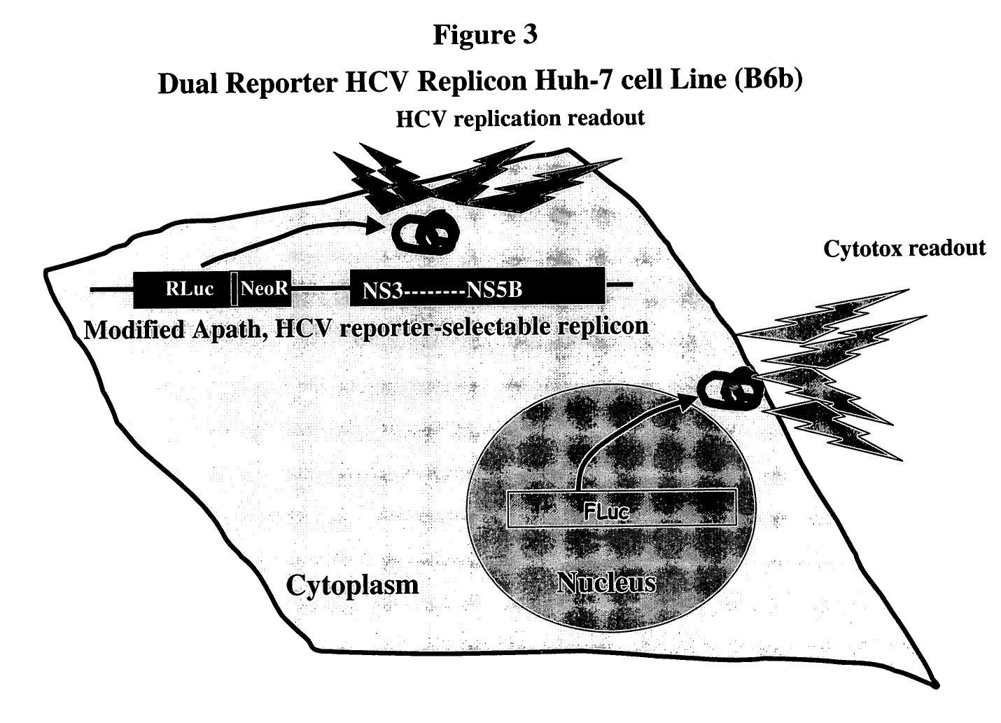

In cell lines containing the BB7M4hRLuc replicon, an increase in replicon RNA by HCV replication results in an increase in hRLuc and NPTII protein production. The high activity of the former can be detected in the replicon cell line by adding a substrate, and the NPTII activity results in stable colony and cell line formation. One of the cell lines that contains the BB7M4hRLuc replicon is BB7#10. This cell line was transformed with pCNA6.FLuc to generate the dual reporter replicon cell line, B6b. B6b as shown in FIG. 3 contains the BB7M4hRLuc replicon that replicates in the cytoplasm and an integrated FLuc gene in the nucleus.

The invention includes the use of a selectable subgenomic HCV replicon RNA that contains a reporter gene and is capable of a high level of replication in the human hepatoma cell line Huh-7, as displayed by a substantial increase in signal-to-noise ratio (S/N) as compared to available reporter-selectable replicons. In particular, a replicon has been constructed for use in the present dual reporter assay, which contains a humanized Renilla luciferase gene separated from a NPTII gene by a self-cleaving peptide of foot and mouth disease virus 2A proteinase. The Huh-7 cell line carrying the reporter-selectable replicon was found to have a stable reporter gene signal over 50 passages and sensitivity to known HCV inhibitors with inhibition values (EC₅₀) comparable to those obtained from other replicon cell lines. This cell line was used for transfection with an expression construct that expresses the firefly luciferase (FLuc) gene (pcDNA6.Fluc). Following selection with G418 and blasticidin (Invitrogen, Carlsbad, Calif.), G418 to select for the replicon and blasticidin to select for integrated FLuc construct, a double-stable cell line, B6b, was created. This cell line expresses hRLuc to monitor antiviral activity and FLuc to monitor cytotoxicity. This dual reporter replicon cell line allows the respective reporters to be used for measuring antiviral activity and cytotoxicity of inhibitors. Furthermore, we have coupled the use of dye reduction methodology to read the hRLuc, FLuc and dye reduction end points from the same well to measure antiviral activity (hRLuc) and cytotoxicity (FLuc/dye reduction) from the same population of cells.

A. Dual Antiviral Activity/Cytotoxicity Reporter Assays Coupled with Dye Reduction Assay Methods

The present assay system , which couples a dual antiviral activity/cytotoxicity reporter assay with another cytotoxicity measurement by the dye reduction methodology, is useful for evaluating the antiviral activity and cytotoxicity of potential drugs in the same population of cells. The assay system is amenable for use in high-throughput formats, while providing rapid and highly quantitative evaluation of the antiviral activities and cytotoxicities of compounds.

The assay system of the invention is useful for compound screening in any system in which compound activity can be monitored a first reporter construct and cell viability can be measured via a second reporter construct. For added confirmation of cytotoxicity, the dye reduction methodology can be used in conjunction with the dual reporter replicon system.

The antiviral activity is measured by monitoring expression of the antiviral reporter. The cytotoxicity reporter, which utilizes a reporter gene different from the compound-reporter, is constitutively expressed in the target cell and is used to measure cell toxicity.

In the dual antiviral activity/cytotoxicity reporter assay system of the invention, target cells can be constructed that constitutively express a reporter gene responsive to cell viability. As a result, the reporter gene is expressed as long as the target cell remains viable and transcriptionally active. Thus, the reporter is a monitor of cytotoxicity.

An HCV reporter replicon cell line was transformed with a reporter construct that was integrated in the host genome. However, a cell line constitutively expressing a reporter gene by its integration into the host genome could also be used as a starting point. This could be subsequently transfected with a reporter-selectable replicon of an RNA virus to generate a double-stable cell line, selected for the cytotoxic reporter integrated in the nucleus and the reporter-selectable replicon replicating in the cytoplasm. After expression, the activity of the reporter genes is measured, in the presence or absence of a compound of interest, using a dual reporter assay method which allows for the measurement of multiple reporter genes in the same population of cells, i.e., in the same well in a microtiter assay plate. Another embodiment of this invention relates to the use of reducing dyes, such as XTT, MTT and WST-1 in the same well where the reporter gene measurement is carried out to determine antiviral activity or cytotoxicity of inhibitors. The use of reducing dyes in the same well as reporter gene measurement for the antiviral and cytotoxic activities enables measurement of cellular proliferation and another end point to measure cytotoxicity of compounds (as described in the examples of this invention) under the same conditions as the other two end points.

In another embodiment of the invention, the dual antiviral activity/cytotoxicity reporter assay system, in conjunction with recording cytotoxicity in the same wells by the dye reduction method, can be used to determine the specific antiviral activity of inhibitors in standard dose response assays using three endpoints. In such assays, target cells containing the reporter-selectable replicon in microtiter are incubated for a desired duration of time at 37° C. and 5% CO₂ with plates containing serial dilutions of test inhibitors or no inhibitor. After the incubation period, the activities of the viral and target cell-encoded reporter genes, as well as cytotoxicity by the dye reduction method, are measured using the appropriate dual reporter assay methods subsequent to the measurement of cellular proliferation by dye reduction. Data from the reporter gene and cellular proliferation measurements can be expressed as the percent of reporter gene activity or optical density (OD) reading in inhibitor-treated cells relative to that of inhibitor-free cells. An analysis of the antiviral component of such data allows for the calculation of the fifty-percent effective concentration (EC₅₀) or ninety-percent effective concentration (EC₅₀) of an inhibitor. In addition, an analysis of the cytotoxicity component of the data can be used to calculate the 50% cytotoxicity concentration (CC₅₀) of an inhibitor. The therapeutic index (TI), which is a measurement of the specific antiviral activity of an inhibitor, can then be calculated by dividing the cytotoxicity (CC₅₀) by the antiviral activity (EC₅₀). The following table illustrates EC₅₀, EC₉₀ and CC₅₀ values generated by the antiviral (hRLuc) and two cytotoxic end points, FLuc and XTT for two commercially available HCV antiviral compounds, interferon alpha (IFN, Sigma Aldrich) and 5,6-dichlorobenzimidazole riboside (DRB, Sigma Aldrich).

TABLE 1
Effect of DRB and IFN on HCV replication and cell
viability in reporter-selectable HCV replicon cell lines

| Cell line | Compound | EC₅₀ | CC₅₀ (XTT) | CC₅₀ (FLuc) | EC₉₀ |
|---|---|---|---|---|---|
| #10 | DRB (uM) | 21.5 | 53.6 | ND | 50.2 |
| B6b | DRB (uM) | 34.1 | 48.7 | 85.1 | 43.5 |
| #10 | DRB (uM) | 33.2 | 48.7 | ND | 58 |
| B6b | DRB (uM) | 53.7 | 40.4 | 60.7 | 55 |
| #10 | IFN (IU/ml) | 0.38 | >10 | ND | 1.4 |
| B6b | IFN (IU/ml) | 0.39 | >10 | >10 | 2.1 |
| #10 | IFN (IU/ml) | 0.67 | >10 | ND | 3.2 |
| B6b | IFN (IU/ml) | 0.43 | >10 | >10 | 2.6 |

#10=hRLuc containing replicon cell line

B6b=FLuc integrated in the nucleus of #10 line

The EC₅₀ values obtained for IFN are consistent with what has been obtained with a TaqMan RNA quantitation assay for this as well as other replicon cell lines (data not shown). The CC₅₀ values for both inhibitors, especially DRB, are similar by either the XTT or FLuc endpoint, validating the use of the FLuc endpoint to measure cytotoxicity.

The dual antiviral activity/cytotoxicity reporter assay system is useful to screen for specific antiviral inhibitors in a high-throughput format. In a high-throughput format, putative inhibitors are added at single or multiple doses to target cells in microtiter plates. In this application, the cell line already contains the reporter-selectable replicon and a gene for monitoring cytotoxicity. At a specified time after incubation, the activities of the viral and target cell-encoded reporter genes are measured in the cells using the appropriate dual assay methods. Data from the reporter gene measurements can then be expressed as the percent inhibition of reporter gene activity in inhibitor-treated reporter replicon containing cells relative to that of inhibitor-free reporter replicon cells. Antiviral activity is then assigned to test inhibitors that (1) effect a significant reduction in the viral-encoded reporter gene activity relative to the no compound control wells and (2) show no significant effect on expression of the target cell-encoded reporter gene relative to the no compound control wells.

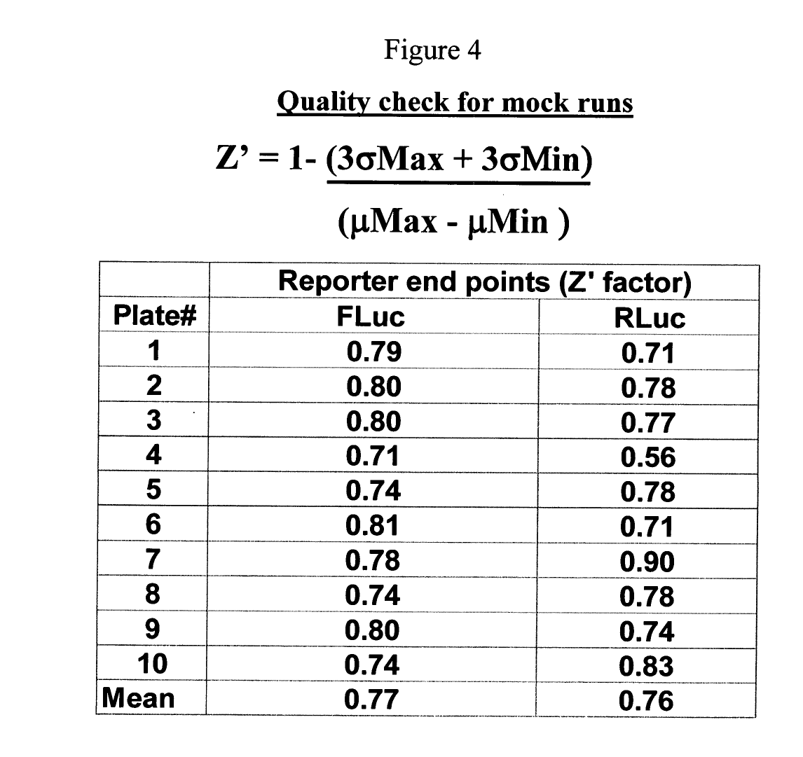

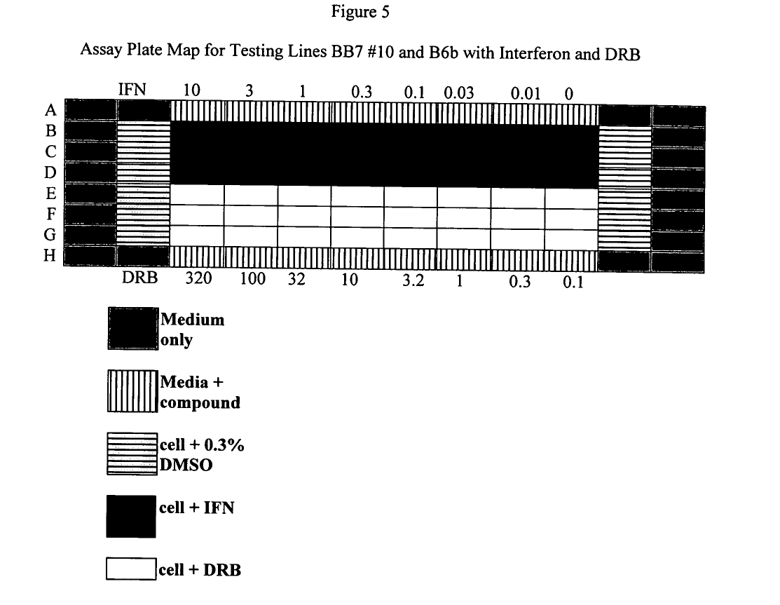

To show that the dual activity/cytotoxicity reporter assays of the invention are amenable for identifying potential drugs in a high-throughput format, coefficients of variation and screening window coefficients (Z′ value) were calculated for the following assay. In 96 well plates a test compound or DMSO was added to dual reporter HCV replicon cell lines expressing humanized Renilla luciferase (RLuc) as the antiviral reporter and a genome integrated firefly luciferase (FLuc) gene as the cytotoxic reporter. Seventy-two hours after incubation at 37° C. and 5% CO₂ the luciferase endpoints were measured. Data from the reporter gene measurements were expressed as the percent inhibition of reporter gene activity in compound-treated cells relative to that of compound-free cells. The antiviral activity and cytotoxicity components of the assay exhibited coefficients of variation (CV) of less than 15%. As shown in FIG. 4, Z′ values of 0.77 and 0.76 were obtained for the FLuc and hRLuc end points, respectively. The Z′ value is reflective of the dynamic range as well as the variation of the assay and is a useful tool for assay comparisons and assay quality determinations (Zhang et al., J. Biomolec. Screen 4:67-73 (1999)). Typically a Z′ value >0.5 is considered favorable for high-throughput screening. Therefore, the low CVs and favorable Z′ values suggest that the HCV replicon dual antiviral activity/cytotoxicity reporter assay is suitable for high-throughput screening.

### Exemplary Methods and Materials

The following examples are given for the purpose of illustrating various embodiments and features of the invention.

### EXAMPLE 1

### Construction of HCV Dual Reporter Replicon Cell Line

The dual reporter replicon cell line, B6b, was constructed by introducing the FLuc gene into the nucleus of the hRLuc reporter-selectable replicon line, BB7M4hRLuc#10. The FLuc gene was cloned into the pcDNA6.1 vector using unique restriction enzyme sites. This vector contains a CMV promoter and the blasticidin resistance gene and the construct is called pcDNA6.Fluc. For transfecting this construct BB7M4hRLuc #10 cells were seeded at 4.1×10⁶ cells in separate T225 tissue culture flasks. The cells were incubated in an incubator at 37° C., 5% CO₂, for approximately 24 hrs. Approximately two flasks were used for each electroporation.

The cells were collected by first removing the media from each flask and washing the cells once with phosphate-buffered saline (PBS). The PBS was then removed by aspirating. Three milliliters of Trypsin-EDTA were added to each flask, making sure that all cells were covered by Trypsin-EDTA and then removed by aspiration. The cells were then incubated at 37° C., 5% CO₂, for 3 min. Seven milliliters DMEM) complete media with 10% FBS (fetal bovine serum), 100 IU/ml of penicillin and 100 mg/ml of streptomycin sulfate (Invitrogen, Carlsbad) were added to each flask. The cell media was mixed by pipeting up and down to suspend the cells evenly. The cells were then transferred to a 50 ml Falcon (Becton Dickinson, Palo Alto) centrifuge tube. The above steps were repeated for all flasks. The cell suspensions were combined in 50 ml centrifuge tubes and centrifuged at 1200 rpm for 5 min. to pellet the cells.

The cells were washed twice in PBS as follows. The media in the tubes was discarded and the cells were resuspended in each tube using 10 ml of PBS. All cells were combined in one 50 ml Falcon centrifuge tube, and PBS was added to generate a final volume of 50 ml. The samples were centrifuged at 1200 rpm for 5 min. The PBS in the tubes was discarded, and the cells were resuspended in the tube using 10 ml of PBS. PBS was again added to generate a 50 ml final volume. The samples were mixed and aliquots were taken to count the cells. The samples were centrifuged at 1200 rpm for 5 min. The PBS in the tube was discarded and the cells were resuspended in PBS (1.0×10⁷ cells/ml) at room temperature (25° C.).

During centrifugation, 10 ml of DMEM complete media was prepared in each 15 ml Falcon centrifuge tube. The pcDNA6.Fluc DNA (1 μg) was added to a sterile microcentrifuge tube on ice. 9 μg of naive Huh-7 total RNA was then added to the microfuge tube. A Bio-Rad Gene PulserII electroporator (Bio-Rad Laboratories, California) was used for electroporation of the plasmid DNA into the BB7M4hRLuc#10 cells, using the following general parameters: 270 V, 950 μF, and 0.4 cm Bio-Rad cuvette.

An aliquot (0.4 ml) of the BB7M4hRLuc#10 cell suspension (see above) was added to one microcentrifuge tube, which contained the DNA sample. The sample was mixed by pipetting up and down several times. The entire DNA-cell mixture was then transferred to a 0.4 cm Bio-Rad cuvette. The electroporator was charged and then discharge pulsed. After the pulse, DMEM complete media from a 15-ml Falcon centrifuge tube (see above) was added immediately, which contained 10 ml of complete media. The mixture was transferred to the same 15-ml Falcon centrifuge tube. The sample was mixed by pipetting up and down, and the entire mixture was transferred to a 100×20 mm tissue culture dish.

The cells were incubated in incubator at 37° C., 5% CO₂, for approximately 24 hrs. The media was replaced with DMEM complete media with 200 μg/ml G418 (Gibco BRL/Invitrogen) and 6 μg/ml Blasticidin (ICN Biomedicals). The cells were incubated in an incubator at 37° C., 5% CO₂, for approximately 34 weeks until the cells were ready for picking colonies or staining. During the incubation, the selective media was replaced once a week.

Once the FLuc construct was electroporated into the BB7 M4hRLuc #10 cells, the plasmid integrated into the genome. Transcription occurs from the CMV promoter, and ultimately results in the translation of firefly luciferase and Blasticidin resistance proteins. Cells were screened for integration of the construct by resistance to blasticidin and the level of FLuc expression. The B6b line was chosen based on the high level of FLuc activity from the nucleus and RLuc signal from the reporter-selectable replicon RNA.

### EXAMPLE 2

### Antiviral Activity and Cell Cytotoxicity (with Dye Reduction Methodology)

Cell line BB7 M4 hRLuc#10 was grown in DMEM without phenol red (catalog # 1053-028) at 10% FBS with 4 mM L-glutamine, 1×Pen-step, 1×non-essential amino acids, and 200 μg/ml G418 (all from Gibco BRL/Invitrogen). Line B6b was grown in the same media with the addition of 6 μg/ml Blasticidin (ICN Biomedicals). Experiments in these replicon lines were carried out in 96-well black wall, clear-bottom plates (Costar®; Corning Incorporated). Cells were seeded as diagrammed in the figure below at a density of 2×10⁴/well in 100 μl DMEM (described above) without G418 or Blasticidin. Cells were allowed to settle at 37° C., 5% CO₂ for 30 minutes. Interferon alpha (IFN) and 5,6-dichlorobenzimidazole riboside (DRB), both obtained from Sigma Aldrich and prepared in 0.6% DMSO, were serially diluted in separate 96 well plates. The concentrations for IFN and DRB ranged from 20 to 0.006 IU/ml and 640 μM to 0.2 μM, respectively (see figure below). One hundred microliters of each concentration was then added to the appropriate well of the cell plate giving a final 1× concentration of 10 to 0.003 IU/ml for Interferon and 320 to 0.1 μM for DRB. Media with 0.6% DMSO was added to the cell only wells in columns 2 and 11; this was equivalent to the final 0.3% DMSO in the highest concentrations of DRB and IFN. The remaining wells bordering the plate were brought up to a final volume of 200 μl with DMEM. The plates were incubated at 37° C., 5% CO₂ for three days.

Cell line BB7 M4 hRLuc #10 contained the selectable reporter replicon BB7 M4-hRLuc, which served as an anti-viral marker, but cytotoxicity had to be measured by XTT. The dual reporter line B6b contained this replicon as well as the firefly luciferase gene, which was integrated into the nucleus for measuring cytotoxicity. In order to validate this line, both XTT and FLuc were used as endpoints to measure cytotoxicity. When utilized in a high-throughput screen, only FLuc will be used as a cytotoxicity end-point.

Processing for XTT determination of cytotoxic concentration 50 (CC₅₀) required addition of 50 μl phosphate buffered saline at pH 7.2 (PBS-Gibco BRL/Invitrogen) containing 1 mg/ml XTT sodium salt and 5 mM phenylmethylsolfonyl fluoride (both from Sigma Aldrich) to all wells. The reducing reaction was incubated for four hours at 37° C. and 5% CO₂. The calorimetric values were obtained by reading on a Kinetic Micro-plate Reader (Molecular Dynamics) at 450/650 nm after manual mixing of the XTT and media. The cells were then processed for reading reporter gene activity. Media/XTT was aspirated from the wells and cells were washed with 100 μl PBS. After removing the PBS, 20 μl of 1× Passive Lysis Buffer (Promega Corp.) was added to each well, and the cells were allowed to lyse at room temperature for 15 minutes. With B6b cell assays, 50 μl of firefly luciferase substrate (Dual Luciferase Kit-Promega Corp.) was used to read reporter activity in a Microbeta Jet 1450 (Wallac Inc). This substrate was added by hand to BB7 M4 hRLuc#10 assays, since the FLuc reporter gene was not present in this cell line. Antiviral activity for both cell lines was then measured using Renilla luciferase.

After addition of the FLuc substrate, 50 μl of RLuc substrate from the Dual Luciferase kit was added, and activity was measured, using the Microbeta Jet. The percent inhibition was calculated after subtracting the background values of media only wells from wells containing cells, and comparing cell only control values to compound wells. The effective concentrations 50 and 90 (EC₅₀ and EC₉₀) of a compound were calculated from the RLuc percent inhibition values, and the CC₅₀ was determined from XTT and/or FLuc inhibition, using Microsoft Excel Fit.

[image]

The foregoing description has been provided to illustrate the invention and its preferred embodiments. The invention is intended not to be limited by the foregoing description, but to be defined by the appended claims.

We claim:

1. A method of evaluating antiviral activity and cytotoxicity of a compound comprising:
(a) providing a target cell population containing a first reporter gene;
(b) introducing a second reporter gene into said cell population by integrating the reporter into a replicon of a positive strand RNA virus and making a dual reporter replicon cell line;
(c) adding a test compound;
(d) incubating said cell population;
(e) measuring the responses of said first and second reporter genes; and
(f) comparing the responses of said first and second reporter genes in cell populations treated with compound to the responses of said first and second reporter genes in cell populations in the absence of said compound.

2. The method of claim 1 wherein the response from said first reporter gene indicates a measure of cell viability, the response from said second reporter gene indicates the activity of a virus, and said second reporter gene is different from said first reporter gene.

3. The method of claim 1 wherein said first reporter gene comprises firefly luciferase and said second reporter gene comprises humanized Renilla reneformis luciferase.

4. The method of claim 1 wherein said first reporter gene comprises humanized Renilla reneformis luciferase and said second reporter gene comprises firefly luciferase.

5. The method of claim 1, wherein said compound comprises an HCV inhibitor.

6. The method of claim 1 wherein the response of said second reporter gene indicates the activity of an RNA virus.

7. The method of claim 1 wherein said cell population is selected from the group consisting of:
Huh-7 cells; HeLa cells; VERO cells; CHO cells; COS cells; BHK cells; HEPG2 cells; 3T3 cells and 293 cells.

8. The method of claim 1 wherein said cell population is contained within a configuration of low-throughput, medium-throughput or high-throughput screening wells.

9. A method of evaluating antiviral activity and cytotoxicity of a compound comprising:
(a) providing a target cell population containing a first reporter gene;
(b) introducing a second reporter gene into said cell population by integrating the reporter into a replicon of a positive strand RNA virus and making a dual reporter replicon cell line;
(c) adding a test compound;
(d) incubating said cell population;
(e) adding a dye reduction agent to said cell population;
(f) measuring the response from the reduced dye reduction agent in said cell population;
(g) comparing the response of said reduced dye reduction agent in a compound treated cell population to the response of said reduced dye reduction agent in cell populations in the absence of the compound;
(h) measuring the expression of the first and second reporter genes; and
(i) comparing the responses of said first and second reporter genes in cell populations treated with compound to the responses of said first and second reporter genes in cell populations in the absence of said compound.

10. The method of claim 9 wherein said cell population is selected from the group consisting of:
Huh-7 cells; HeLa cells; VERO cells; CHO cells; COS cells; BHK cells; HEPG2 cells; 3T3 cells and 293 cells.

11. The method of claim 9 wherein said cell population is contained within a configuration of low-throughput, medium-throughput or high-throughput screening wells.

12. A double-stable, double-reporter, transformed mammalian cell line wherein a first transformation is due to integration of a gene in the nucleus and a second transformation is due to the replication of a RNA virus replicon.

13. The cell line of claim 12 wherein said first and second transformations incorporate the expression of reporter genes to produce a double-stable, double-reporter cell line.

14. The cell line of claim 12 wherein said second transformation is due to the replication of an HCV replicon.

15. A transformed cell line comprising a reporter construct wherein said construct expresses a marker for monitoring cytotoxicity.

16. A method for evaluating both antiviral activity and cytotoxicity of a compound in the same population of cells comprising the steps of:
(a) providing a target cell comprising a reporter gene that is indicative of the activity of an HCV or other RNA virus replicon;
(b) adding a test compound to said population of cells;
(c) incubating said cell population;
(d) adding a dye reduction agent to said cell population;
(e) measuring the response from the reduced dye reduction agent in said cell population;
(f) comparing the response of said reduced dye reduction agent in a compound treated cell population to the response of said reduced dye reduction agent in cell populations in the absence of the compound;
(g) measuring the expression of the reporter gene integrated in the replicon; and
(h) comparing the responses of said reporter gene of the replicon in cell populations treated with compound to the responses of the reporter gene of the replicon in cell populations in the absence of said compound.

17. A method for generating a double-stable, double-reporter cell population comprising:
(a) integrating a luciferase reporter gene into the cell nuclei of said cell population and
(b) introducing an HCV replicating virus sequence into said cell population.


SEQUENCE LISTING (1 sequence)

SEQ ID NO: 1 - DNA, 6618 bp, Artificial
  pcDNA6.Fluc Reporter Construct
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc